(19) United States
(12) Patent Application Publication
(10) Pub. No.: US 2003/0092613 A1
(43) Pub. Date: May 15, 2003

(54) Alpha7 nicotinic receptor peptides as ligands for beta amyloid peptides

(76) Inventors: Daniel H. S. Lee, Sudbury, MA (US); Hoau-Yan Wang, Philadelphia, PA (US); Carlos Plata-Salaman, Ambler, PA (US); Allen B. Reitz, Lansdale, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/928,636

(22) Filed: Aug. 13, 2001

Related U.S. Application Data
(60) Provisional application No. 60/225,048, filed on Aug. 14, 2000.

(51) Int. Cl. A61K 38/16; A61K 38/10; A61K 38/08; C07K 14/435; C07K 7/08; C07K 7/06

(52) U.S. Cl. 514/12; 514/13; 514/14; 514/15; 514/16; 514/17; 514/18; 530/324; 530/326; 530/327; 530/328; 530/329; 530/330

(57) ABSTRACT

The present invention describes native and degenerate peptides derived from human α7 nicotinic receptor useful as minimized ligands for β amyloid peptides. These peptides are useful to discover compounds that inhibit the interaction with β amyloid peptides with the α7 nicotinic receptor, and are also useful in assays to measure β amyloid.

### CROSS REFERENCE OF RELATED APPLICATIONS

This application claims priority from U.S. provisional application Serial No. 60/225,048, filed Aug. 14, 2000, which is hereby incorporated by reference.

### FIELD OF THE INVENTION

The present invention describes native and degenerate peptides derived from human α7 nicotinic receptor useful as minimized ligands for β amyloid peptides. These native and degenerate peptides derived from human α7 nicotinic receptor are useful to discover compounds that inhibit the interaction of β amyloid peptides with the α7 nicotinic receptor, are also useful in assays to measure the levels of β amyloid peptides. Further the native and degenerate peptides derived from human α7 nicotinic receptor are potentially therapeutically useful for the treatment of Alzheimer's disease and related neurodegenerative disorders

### BACKGROUND OF THE INVENTION

Neurodegenerative disorders such as Alzheimer's disease (AD) and Parkinson's disease (PD) afflict humanity with great suffering and financial loss. AD is characterized by neurofibrillary tangles, neuritic plaques, and neuronal cell death. AD appears as either the familial, early onset or late-onset forms, with the latter being more prevalent. AD is the major cause of age-related dementia and cognitive impairment (Wisniewski, T.; Ghiso, J.; Frangione, B. Neurobiol. of Disease 1997, 4, 313-328). Currently the primary means for the diagnosis of AD consists of monitoring the severely diminished cognitive functioning of the patient. There is a need to develop sensitive, biochemical methods to determine the prognosis and progression of the disease, and to monitor the therapeutic efficacy of treatment given to subjects likely to develop or who are presently suffering from Alzheimer's disease.

The neurotoxic β-amyloid peptide₁₋₄₂ [Aβ₁₋₄₂] is abundantly present in the amyloid plaques of Alzheimer's disease (AD) brains and also modulates cholinergic functions which are critical in memory and cognitive neurophysiology (Auld, D. S., Kar, S., Quirion, R. Trends Neurosci 1998; 21: 43-49). Aβ₁₋₄₂ interacts selectively with high affinity to the neuronal pentameric cation channel α7 nicotinic acetylcholine receptor (Wang, H. -Y. et al. J Biol. Chem. 2000, 275, 5626). This peptide-receptor interaction is likely neurotoxic due to the observation that stimulation of the α-7 subtype of the nicotinic acetylcholine receptors (nAChRs) can protect neurons against Aβ cytotoxicity (Kihara, T. et al. Ann. Neurol. 1997, 42, 159). Also, compounds that activate nAChRs, especially of the α-7 subtype, have been found to have in vivo activity in models of cognition enhancement (U.S. Pat. No. 5,741,802, issued Apr. 21, 1998). The α7 nicotinic acetylcholine receptor is involved in calcium homeostasis and modulation of acetylcholine release in the synapses, all of which are critical events in cognitive function. β-Amyloid peptide increases cytosolic-free Ca²⁺ in AD lymphoblasts (Ibarreta et al., Alzheimer Dis Assoc Disord 1997 December; 11 (4):220-7), and elevates mitogen-induced Ca²⁺ responses in freshly prepared human lymphocytes (Eckert et al., Life Sci 1994;55(25-26):2019-29). Amyloid precursor protein (APP) can be induced on the cell surface of human lymphocytes upon stimulation (Bullido et al., Biochim Biophys Acta 1996 August 21; 1313(1):54-62) and increased APP-770 isoform is observed upon examination of the lymphocytes from AD patients (Ebstein et al., Brain Res Mol Brain Res 1996 January;35(1-2):260-8). Lymphoblastoid cells from patients with early-onset and late-onset familial AD show increased expression of APP mRNA and protein (Matsumoto et al., Eur J Biochem Oct. 1, 1993 ;217(1):21-7). The interaction of Aβ₁₋₄₂ and the α7 nicotinic acetylcholine receptor may initiate a series of pathophysiological events observed in AD and result in neurotoxicity and cognitive dysfunction, a hallmark of AD. Investigators have measured α7 nicotinic acetylcholine receptor mRNA levels in the brains of normal and patients with AD in order to determine changes in regional distribution or changes in the total amount of mRNA levels between the subject populations. α7 Nicotinic acetylcholine receptor mRNA was equally distributed in all areas of the brain except the hippocampus, where AD subjects exhibited higher mRNA levels than the control population. Similarly, lymphocytes from AD patients exhibit an increased mRNA level for α7 nicotinic acetylcholine receptor (Hellstrom-Lindahl et al., Brain Res Mol Brain Res 1999 March 20;66(1-2):94-103). In a second report, nicotinic acetylcholine receptor binding in the brains of AD and control subjects showed no significant difference (Lang, W. and Henke, H. Brain Res 1983; 267: 271-280). It is generally believed that the cognition and memory deficits observed in Alzheimer's disease patients are the results of the adverse effects of Aβ₁₋₄₂ on the cholinergic neurons (Kar, S. et al J. Neurochem. 1998; 70, 2179-2187). We have shown that β-amyloids bind with high affinity to the human pentameric α7 nAChR (2-site fit) that may be critically and intimately involved in the pathogenesis of Alzheimer's disease (Wang, H. -Y. et al. J. Biol Chem. 2000; 275, 5626-5632.). We have also shown that the α7nAChR protein levels decreased in Alzheimer's disease brains when compared to age-matched non-demented controls, which may be useful in the development of a biochemical assay for diagnosis or disease progression monitoring (WIPO publication WO9962505 by Reitz et al and published Dec. 9, 1999).

The development of high throughput drug discovery binding assays using ion channel receptors such as the α7nAChR has been difficult because of issues arising from rapid ion channel desensitization and low receptor density on the membranes, even in recombinant cells. Recent work has shown that a peptide stretch deriving from the rodent nAChRs maintained the full binding characteristics of native nAChRs to small molecule ligands (Lentz, T. Biochem. Biophys. Res. Commun. 2000; 268, 480-484), including the two binding sites. In the present invention we describe native and degenerative peptides originating from the human α7 nAChR that not only maintain the binding characteristics of native α7 nAChR, including binding to the β-amyloids, but also modulate β-amyloid-mediated physiology. These peptides are useful in assays to discover compounds that inhibit the interaction of β amyloid peptides with the α7 nicotinic receptor, and are also useful in assays to measure β amyloid.

U.S. Pat. No. 5,837,489 describes the human α7 nicotinic receptor, but is silent regarding the use of fragments thereof as ligands for β amyloid peptides. The present disclosure is the first known report of α7 nicotinic receptor derived peptides as ligand for β amyloid peptide binding.

### SUMMARY OF THE INVENTION

The present invention is directed to methods for diagnosing Alzheimer's disease involving the binding of Aβ to huma7 that allows the measurement of Aβ₁₋₍₄₀₎₄₂ and related peptides in the biological samples. The present invention also describes a method for designing a drug discovery assay for identifying α7 nAChR modulators based on the interaction of huma7 with Aβ. Finally, fragments of huma7 may provide a therapeutic agent that attenuates the biological effects of Aβ₁₋₄₂ in AD.

The present invention is further directed to β-amyloid binding peptides comprising a sequence selected from a group consisting of

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| SERFYECCKEP, | (SEQ.ID.NO.:9) |
| AERFYECCKEP, | (SEQ ID No.:10) |
| SARFYECCKEP, | (SEQ ID No.:11) |
| SERAYECCKEP, | (SEQ ID No.:12) |
| SERFAECCKEP, | (SEQ ID No.:13) |
| SERFYACCKEP, | (SEQ ID No.:14) |
| SERFYEACKEP, | (SEQ ID No.:15) |
| SERFYECCAEP, | (SEQ ID No.:16) |
| SERFYECCKEA, | (SEQ ID No.:17) |
| GIPGKRSERFYECCK, | (SEQ ID No.:18) |
| SERFYECCKEP, | (SEQ ID No.:19) |
| YECC, | (SEQ ID No.:20) |
| HSVTYSCCPDT, | (SEQ ID No.:21) |
| NTRKYECCAEI, | (SEQ ID No.:22) |
| NTRKYECCAEI, | (SEQ ID No.:23) |
| AVGTYNTRKYECCAEIYPDI, | (SEQ ID No.:24) |
| NGEWDLVGIPGKRSERFYECCKEPYPDVTFTV, | (SEQ.ID.NO.:25) |
| FYEC, | (SEQ.ID.NO.:26), |
| SGYIPNGEWDLVGIPGKRSERFYECCKEPYPD, | (SEQ.ID.NO.:27) |
| and | |
| ECCK. | (SEQ.ID.NO.:28) |

### BRIEF DESCRIPTION OF THE DRAWINGS

FIG. 1—Huma7 peptide inhibits ¹²⁵I-β amyloid binding to α7-SK-N-MC membranes. Triangle=tetrapeptide YPWF Square=Inhibitor A Circle=Huma7 peptide.

FIG. 2—Huma7 peptide inhibits ¹²⁵I-β amyloid binding to an immobilized huma7 peptide and related peptide derivatives. (H7201-220=SEQ.ID.NO.:6; H7200-214=SEQ.ID.NO.:4; H7201-218=SEQ.ID.NO.:5; H7206-216=SEQ.ID.NO.:7; Huma7=SEQ.ID.NO.:25)

FIG. 3—Huma7 peptide inhibits Fluorescent-β amyloid₁₋₄₂ deposition onto synthaloid plates.

FIG. 4—Fluo-Aβ₁₋₄₂ binding to Biotin-Huma7-5 peptide.

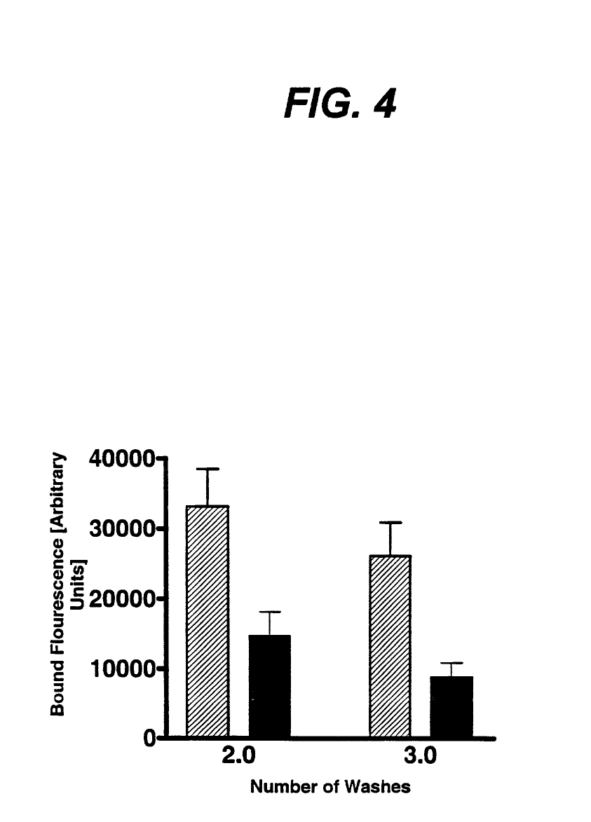

FIG. 5—Time course for ¹²⁵I-Aβ₄₀ binding to biotinylated huma7-5 peptide showing saturation at about 6 hours.

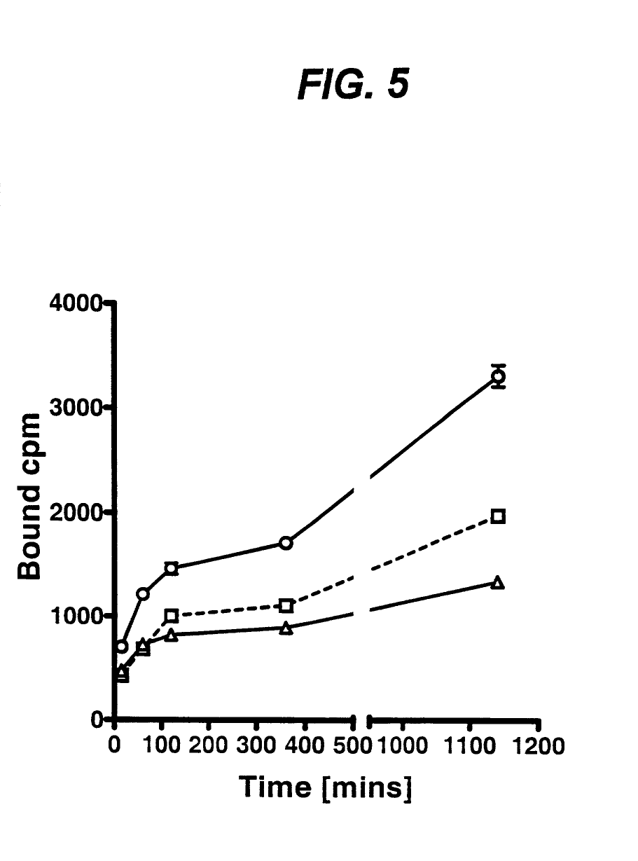

FIG. 6—Inhibition of ¹²⁵I-Aβ₄₀ binding to biotinylated huma7-5 by huma7-5.

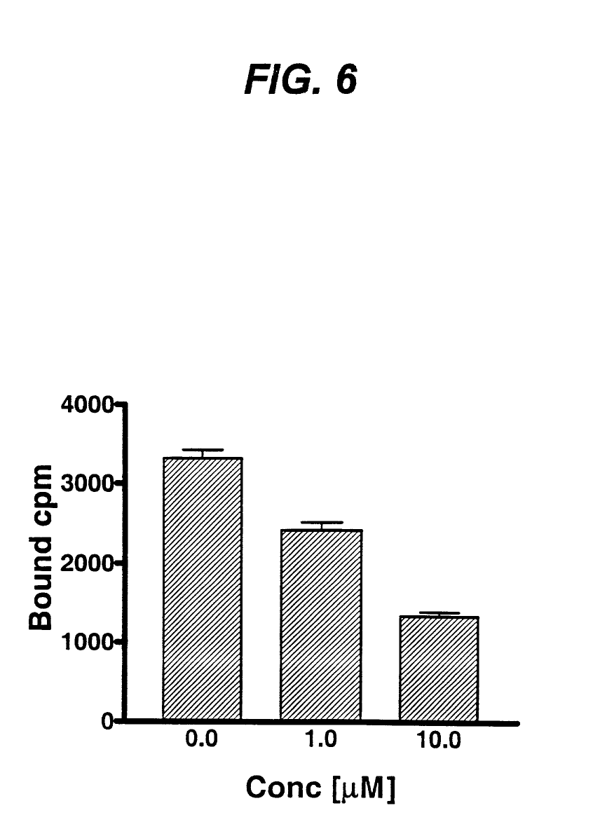

### DETAILED DESCRIPTION OF THE INVENTION

Definitions

As used herein the term “β amyloid peptide” is a β-amyloid peptide₁₋₄₂, β-amyloid peptide_{1-40,} or enzymatically modified β-amyloid peptide, such as where aspartic acid at position one is modified to isoaspartic acid or where the glutamic acid at position three is converted to a pyroglutamyl residue with excision of residues 1 and 2 (Aβ3pE). Particularly preferred Aβ peptides are selected from the group consisting of β-amyloid₁₋₄₂ and Aβ3pE.

The term “subject” as used herein, refers to an animal, preferably a mammal, most preferably a human, who has been the object of treatment, observation, biochemical screening, or experiment.

β Amyloid Binding Peptides

The present invention provides peptides derived from the human α7 nAChR that bind to β amyloid peptides. The identification and characterization of these peptides allows the development of a method for diagnosing Alzheimer's disease (AD) in a subject and a method for screening small molecules for treating Alzheimer's disease. Specifically, these peptides can be used in a method for diagnosing AD by binding to β amyloid thus enabling the measurement of free β-amyloid₁₋₄₂ or β-amyloid₁₋₄₀ present in biological tissues or fluids in AD patients, or those who are in the early stages of AD, or who will develop AD in the future. The binding reaction between these peptides and β amyloid can also be employed in the development of drug screening assays, particularly high throughput assays, for the identification of small molecule α7 nAChR modulators for treating various disorders including, but not limited to neurodegenerative disorders such as Alzheimer's disease and related disorders, Parkinson's disease, pain, neuropathic pain, depression, generalized anxiety disorder, obsessive compulsive behavior, phobias, post-traumatic stress disorder, panic disorder, schizophrenic disorders and psychosis, bipolar disorder, dementia, and substance abuse.

The peptides of the present invention can also be therapeutically active agents, or can be used to model interactions with β amyloids for the purpose of rationale drug design to create small molecule mimetics using computer-based molecular modeling. These computer-based methods fall into two broad classes: database methods and de novo design methods. In database methods the compound of interest is compared to all compounds present in a database of chemical structures and compounds whose structure is in some way similar to the compound of interest are identified. The structures in the database are based on either experimental data, generated by NMR or x-ray crystallography, or modeled three-dimensional structures based on two-dimensional (i.e., sequence) data. In de novo design methods, models of compounds whose structure is in some way similar to the compound of interest are generated by a computer program using information derived from known structures, e.g., data generated by x-ray crystallography and/or theoretical rules. Such design methods can build a compound having a desired structure in either an atom-by-atom manner or by assembling stored small molecular fragments.

The present invention is directed to β-amyloid binding peptide comprising a sequence selected from the group consisting of

| | |
|---|---|
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| SERFYECCKEP, | (SEQ.ID.NO.:9) |
| AERFYECCKEP, | (SEQ ID No.:10) |
| SARFYECCKEP, | (SEQ ID No.:11) |
| SERAYECCKEP, | (SEQ ID No.:12) |
| SERFAECCKEP, | (SEQ ID No.:13) |
| SERFYACCKEP, | (SEQ ID No.:14) |
| SERFYEACKEP, | (SEQ ID No.:15) |
| SERFYECCAEP, | (SEQ ID No.:16) |
| SERFYECCKEA, | (SEQ ID No.:17) |
| GIPGKRSERFYECCK, | (SEQ ID No.:18) |
| SERFYECCKEP, | (SEQ ID No.:19) |
| YECC, | (SEQ ID No.:20) |
| HSVTYSCCPDT, | (SEQ ID No.:21) |
| NTRKYECCAEI, | (SEQ ID No.:22) |
| NTRKYECCAEI, | (SEQ ID No.:23) |
| AVGTYNTRKYECCAEIYPDI, | (SEQ ID No.:24) |
| NGEWDLVGIPGKRSERFYECCKEPYPDVTFTV, | (SEQ.ID.NO.:25) |
| FYEC | (SEQ.ID.NO.:26) |
| SGYIPNGEWDLVGIPGKRSERFYECCKEPYPD | (SEQ.ID.NO.:27) |
| and | |
| ECCK. | (SEQ.ID.NO.:28) |

Preferred β amyloid binding peptides are those comprising a sequence selected from the group consisting of

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| cyclic peptide | |
| SERFYECCKEP, | (SEQ.ID.NO.:9) |
| YECC, | (SEQ.ID.NO.:20) |
| huma7 | |
| NGEWDLVGIPGKRSERFYECCKEPYPDVTFTV, | (SEQ.ID.NO.:25) |
| FYEC | (SEQ.ID.NO.:26) |
| SGYIPNGEWDLVGIPGKRSERFYECCKEPYPD | (SEQ.ID.NO.:27) |
| and | |
| cyclic peptide | |
| ECCK. | (SEQ.ID.NO.:28) |

Particularly preferred β amyloid binding peptides are those comprising a sequence selected from the group consisting of

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| cyclic peptide | |
| SERFYECCKEP, | (SEQ.ID.NO.:9) |
| YECC, | (SEQ.ID.NO.:20) |
| huma7 | |
| NGEWDLVGIPGKRSERFYECCKEPYPDVTFTV- | (SEQ.ID.NO.:25) |
| amide | |
| and | |
| FYEC. | (SEQ.ID.NO.:26) |

The peptides of the present invention are preferably N-acetylated and C-terminal amidated. The peptides of the present invention may be synthetically produced using methods well known in the art utilizing D or L isomeric amino acid precursors. Therapeutic peptides of the present invention preferably contain one, a few, or all of their amino acid residues in the D-isomer conformation. Cyclic peptides are indicated by underline of the relevant amino acids. Cyclic peptides are those where the two adjacent cystines in the sequence were oxidized to form the disulphide bridge. We have used the term ‘cyclic’ to differentiate from the linear sequence that does not contain the disulphide bond.

The peptides of the present invention may also be produced by recombinant technology, as small peptides, as small synthetic fusion proteins comprising one or more peptides of the present invention fused to a targeting peptide. The term “fusion protein” as used herein refers to protein constructs that are the result of combining multiple protein domains or linker regions for the purpose of gaining function of the combined functions of the domains or linker regions. This is most often accomplished by molecular cloning of the nucleotide sequences to result in the creation of a new polynucleotide sequence that codes for the desired protein. Alternatively, creation of a fusion protein may be accomplished by chemically joining two proteins together. The term “linker region” or “linker domain” or similar such descriptive terms as used herein refers to stretches of polynucleotide or polypeptide sequence that are used in the construction of a cloning vector or fusion protein. Functions of a linker region can include introduction of cloning sites into the nucleotide sequence, introduction of a flexible component or space-creating region between two protein domains, or creation of an affinity tag for specific molecule interaction. A linker region may be introduced into a fusion protein without a specific purpose, but results from choices made during cloning. Targeting peptides allow directed localization of the peptide to a cellular organelle or delivery of the peptide into the secretory pathway of the cell. The peptides of the present invention may also be a component in a fusion gene. For example the peptides of the present invention may be expressed as in phage display or as a replacement in the variable loop of an immunoglobulin fold. Hence the present invention provides fusion proteins containing a β amyloid binding sequence selected from a group consisting of:

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| cyclic peptide SERFYECCKEP, | (SEQ.ID.NO.:9) |
| YECC, | (SEQ.ID.NO.:20) |
| huma7 | |
| NGEWDLVGIPGKRSERFYECCKEPYPDVTFTV, | (SEQ.ID.NO.:25) |
| FYEC, | (SEQ.ID.NO.:26) |
| SGYIPNGEWDLVGIPGKRSERFYECCKEPYPD | (SEQ.ID.NO.:27) |
| and | |
| cyclic peptide | |
| ECCK, | (SEQ.ID.NO.:28) |

operably linked to another sequence, with the proviso that the present invention excludes fusion proteins that function as α7 nicotinic acetylcholine receptors.

Drug Screening Assays

The peptides of the present invention are useful to screen for putative inhibitors of β amyloid/α7 nicotinic acetylcholine receptor interaction. The assay comprises the steps:

(a) contacting a β amyloid peptide, a β amyloid binding peptide, and a putative inhibitory compound for sufficient time to provide a β amyloid/binding peptide complex; and

(b) measuring a change in quantity of β amyloid/binding peptide complex.

The preferred drug screening assays of the present invention utilize an easily measurable marker of interaction, for example radiolabeled β amyloid or binding peptide or a fluorescent derivative of either peptide. Measurement is made using methods well known in the art including radionucleotide decay, fluorescence intensity, fluorescent resonance energy transfer (FRET), or anisotropy, mass spectrometry, or an α7nAChR ligand labelled appropriately, e.g. ¹²⁵I-α-bungarotoxin.

The term “putative inhibitory compound” as used herein refers to an organic molecule that has the potential to disrupt the interaction between the β amyloid and the β amyloid binding peptide. For example, but not to limit the scope of the current invention, compounds may include small organic molecules, other synthetic or natural amino acid peptides, proteins, or synthetic or natural nucleic acid sequences, or any chemical derivatives of the aforementioned. Particular examples of compounds useful in the methods of the present invention include certain compounds described in WO9962505 by Reitz et al and published on Dec. 9, 1999.

The compound described as inhibitor A has the structure

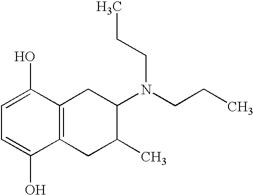

and is described as “compound 9” in WIPO publication WO99/62505 by Reitz et al.

Diagnostic Assays

The peptides of the present invention are also useful in diagnostic assays to detect the presence of β amyloid in a biological sample. A “biological sample” as used herein, refers to a biological substance that contains a β Amyloid peptide, such as red blood cells, white blood cells, platelets, ascites, urine, saliva, olfactory neuroepithelia, skin fibroblasts, cerebrospinal fluid, plasma, serum and other constituents of the body that may contain the β Amyloid peptide. Further, a sample may be a component in a larger composition, for example in a tissue section of a biopsy, where the sample may be an unisolated fraction of biological fluid or one or more cellular subtypes amongst a field of different cell types.

The diagnostic assay of the present invention comprise the steps:

(a) contacting a biological sample with a β amyloid binding peptide;

(b) isolating a β amyloid peptide with the binding peptide; and

(c) detecting the β amyloid peptide.

Alternatively, the peptides of the present invention can be used as a labeled reagent to detect β amyloid isolated by other methods, for example using a β amyloid specific antibody.

The levels of β amyloid peptide are measured by methods known in the art including, but not limited to, immunoassay techniques, HPLC analysis, HPLC/MS (mass spectral) analysis, MALDI/TOF (matrix-assisted laser desorption/time-of-flight) mass spectral analysis, size exclusion chromatography, thioflavin-T or Congo Red staining, or ES/MS (electrospray ionization mass spectral) analysis. By comparing the levels of β-amyloid₁₋₄₂ or Aβ3pE with normal (control) patients, or by comparing the levels of β-amyloid₁₋₄₂ or Aβ3pE in any particular patient over time, one of ordinary skill in the art can determine whether a patient is suffering from AD or is at risk for developing AD, and one can monitor the progression of AD.

A particularly preferred assay format is the affinity capture assay format, wherein the β amyloid peptide is isolated using one affinity capture reagent, and is detected with an affinity label reagent, with the proviso that one of these affinity reagents is a β amyloid binding peptide of the present invention. The affinity capture or affinity label reagents comprise compounds capable of specific interaction with the β amyloid peptide to the exclusion of similar compounds. These compounds include, for example, synthetic or natural amino acid polypeptides, proteins (including antibodies), small synthetic organic molecules, or deoxy- or ribo-nucleic acid sequences with about 20-fold or greater affinity for the β amyloid peptide compared to other proteins or peptides.

Also included in the invention is a diagnostic kit in which all of the components required for a viable diagnostic determination are packaged together sufficient for isolation of β amyloid peptide from a sample and subsequent analysis of the levels of the β amyloid peptide such as by an ELISA (enzyme linked immunosorbant assay). The components of an immunoassay diagnostic kit include an affinity capture reagent, for instance an affinity coated resin, an affinity label reagent, wherein one of the affinity reagents is a β amyloid binding peptide of the present invention, and optionally immunoassay control reagents. Immunoassay control reagents are those that confirm proper function of the assay system and serve to validate interpretation of the sample. A negative control is one that yields no signal from the affinity label reagent, and is often used to determine the background noise of an assay system or used to calculate the signal to noise ratio for a positive sample using calculations well known in the art. A positive control is one that yields a signal from the affinity label reagent, and is often used to validate the assay system or to compare to a sample to interpret the status of the sample. This can be done empirically using a “yes/no” system or can be made quantitative by comparing the signal generated by control samples containing known quantities of β amyloid peptide with a test sample.

The following examples illustrate the present invention without, however, limiting the same thereto. As used herein, the abbreviation “α-BTX” shall mean α-bungarotoxin

### EXAMPLE 1

### B Amyloid Binding Peptide Competion with Alpha7 Nachr Interaction with β Amyloid Peptide

α7nAChR (nicotinic acetylcholine receptor) binding assay: Membranes from recombinant SK-N-MC cells (ATCC#HTB-10) overexpressing α7nAChR (α7SK-N-MC) were prepared according to standard procedures (Wang et al, J. Biol Chem. 275, 5626-5632, 2000) except that the membranes were thoroughly washed. For each assay, membranes (20 μg) were precoupled to wheat germ agglutinin yittrium SPA beads (250 μg, Amersham) in 50 mM HEPES (pH 7.5) containing 0.01% BSA, 2 mM Mg²⁺, 2 mM Ca²⁺ and protease inhibitors (Boehringer Mannheim, Germany). The inhibitors including huma7 and Inhibitor A (a small organic molecule that inhibits β amyloid folding) were then added to the assay mixture, followed by ¹²⁵I-β-amyloid₁₋₄₀ (20 pM, 2000 Ci/mmol, Amersham) to a final volume of 150 μl. At the end of the incubation (30 min), bound radioactivity was measured in a TopCount (Packard, Meriden, Conn.). Data were analyzed using Graphpad Prism software (Graphpad Software Incorporated).

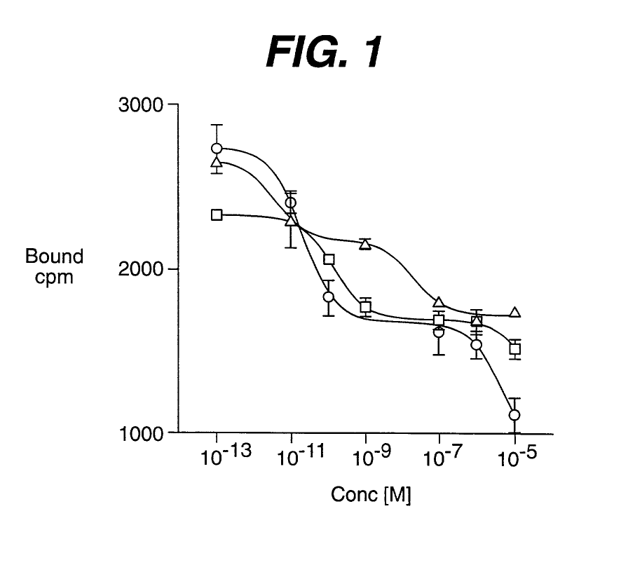

Huma7 peptide potently inhibited ¹²⁵I-β-amyloid₁₋₄₀ binding to α7SK-N-MC membranes suggesting that it competes with native receptor for β amyloid ligand (FIG. 1 and Table 1).

TABLE 1
Calculated EC50 for biphasic response

| | YPWF peptide | Inhibitor A | HUMA7 |
|---|---|---|---|
| EC₅₀ - 1 | 3.513 × 10⁻¹² | 1.332 × 10⁻¹⁰ | 2.015 × 10⁻¹¹ |
| EC₅₀ - 2 | 2.027 × 10⁻⁸ | 3.47 × 10⁻⁵ | 4.876 × 10⁻⁶ |

One possibility considered for huma7 to display this inhibitory effect is that the binding of peptide itself to the ¹²⁵I-β-amyloid₁₋₄₀ ligand. This hypothesis was tested in the following assay where the peptide was first immobilized and then allowed to interact with the ligand using a solid-phase adsorption affinity assay.

Huma7 binding assay: Huma7 peptide, derivatives of huma7 peptide, or BSA (bovine serum albumin (1 μg/well) were immobilized onto the surface of a Polysorb 96-well plate. Unbound peptide was removed and ¹²⁵I-β-amyloid₁₋₄₀ (40 pM, 2000 Ci/mmol, Amersham) was added to a final volume of 100 μl in the presence of 10 μM inhibitors such as a tetrapeptide YPWF, huma7, α-Bungarotoxin, and “inhibitor A”. After incubation at 25° C. for 1 hour, unbound ligand was removed, 25 μl of Microscint was added to each well and the bound radioactivity was measured in a B-plate counter.

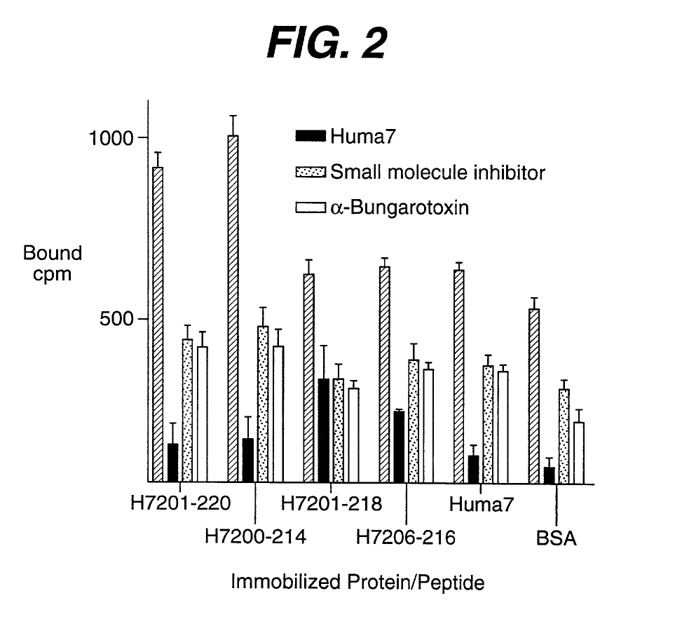

The results showed that huma7 bound to the ¹²⁵I-β-amyloid₁₋₄₀ ligand and the binding can be inhibited by the α7nAChR antagonist, α-bungarotoxin, or Inhibitor A (FIG. 2) suggesting that huma7 contains the binding motifs for β-amyloids. Therefore the huma7 peptide likely inhibits β amyloid binding to native receptor by competitive inhibition.

β-Amyloid deposition assay: β-Amyloid deposition on the Synthaloid plate (Biosource International, Camarillo, Calif.) was carried out as previously described (Esler, W. P. et al Nature Biotech 1997; 15, 258-263) with minor modifications. Fluo-β-amyloid₁₋₄₂ [Advanced BioConcepts, Montreal, Quebec, 50 nM] in 100 μl of 10 mM HEPES, pH 7.4 containing 0.001% sodium azide, 0.1% ovalbumin and protease inhibitors were added to each well of the Synthaloid plate followed by the addition of inhibitors and incubated at 25° C. for 5 hr. The wells were washed 3 times to remove unbound fluo-β-amyloid. Bound fluorescence representing deposited β-amyloid was measured in the LJL Analyst fluorescence reader (LJL BioSystems, Sunnyvale, Calif.).

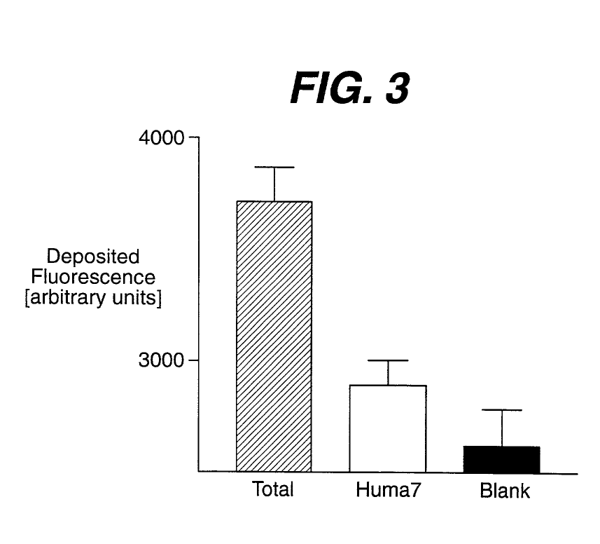

The ability of huma7 to inhibit β-amyloid deposition on the Synthaloid plate was measured, and as expected, huma7 inhibited β-amyloid deposition in vitro (FIG. 3).

### EXAMPLE 2

### Biological Assays Using Beta Amyloid Binding Peptides Varients

ACh release from guinea pig hippocampal synaptosomes: Synaptosomes (P2 fraction) were prepared from guinea pig hippocampus as described previously and re-suspended in a Krebs-Ringer solution containing 0.1 μM [methyl-³H]choline chloride (NEN, Boston, Mass.) and incubated at 37° C. for 30 min. Unincorporated choline was removed by washing and the synaptosomes were resuspended in Krebs-Ringer solution to 10 mg/mL. ACh release from synaptosomes was measured using the parallel superfusion chambers (Hugo Sachs Elektronik, March-Hugstetten, Germany) according to that described earlier (Wang, H. -Y. et al Neuropeptides 1999; 33, 197-205). The synaptosomal suspension (250 μL) was placed in each of the superfision chambers between two circular pieces of GF/B filters (Whatman, England). The chambers were perfused with oxygenated Krebs-Ringer containing 1 μM physostigmine hemisulfate at a flow rate of 0.5 mL/min. The onset of superfusion was defined as time zero (to). Starting 30 min after t₀, fractions of the effluent were collected 12 times at 10-minute intervals.

The tissues were treated with peptides and ACh release was evoked by superfusing with 65 mM K⁺-Krebs-Ringer (made by equimolar replacement of NaCl with KCl) for 30 seconds. No significant deterioration in fractional release was observed during 4 consecutive depolarization stimuli (S1, S2, S3 and S4) delivered at 30-minute intervals. Twenty minutes prior to K⁺ stimulation (S2, S3 or S4), tissues were superfused with Krebs-Ringer solution that contained various concentrations of compound or vehicle (control) for 30 min. After perfusion, 5 mL of Scintsafe PlusTM 50% (Fisher Scientific) was added to the synaptosomes (on filter) and aliquots of the superfusates (0.5 mL). The amount of ³H in the superfusate samples was measured by liquid scintillation spectrometry.

Tritiurn efflux into the superfusate was calculated as the fraction of ³H content in the synaptosomes at the onset of the respective collection period. To calculate the amount of ³H present in the superfusate after K⁺ stimulation, the estimated basal efflux (the average of the 10-minute fractions immediately before and after each stimulation) was subtracted from the total efflux during the 10-minute fraction which began with the 30-second second K⁺ stimulus. This difference was then used to calculate the percent of K⁺-evoked release of ACh.

In order to quantify the in vitro effect of a compound e.g. huma7 in the presence of 1 nM β-amyloid₁₋₄₂ on K⁺-stimulated ³H release, the ratio of the fraction released by a given K⁺ stimulus, Sn (S2, S3 or S4) to that evoked by the control stimulus (S1), was determined. Data are expressed as the mean fractional ³H release±S.E.M. Statistical differences between treatments were tested by Student's t test, two-tailed or the two-factor ANOVA as appropriate. Individual differences in the dose-response curve were evaluated by the Newman-Keuls multiple comparison test.

The efficacy of huma7 to interact with β-amyloids was demonstrated by the ACh release assay using animal cortical synaptosome preparations. Huma7 efficiently blocked the β-amyloid-induced inhibition of potassium-evoked ACh release (Table 2).

TABLE 2
Huma7 and its peptide fragments attenuated β-amyloid's
inhibition on potassium-evoked ACh release
from rodent synaptosomes

| | | | ACh |
|---|---|---|---|
| SEQ. | | Inh ACh | rel % |
| ID. | | rel IC₅₀ | Inh @ |
| NO.: | Sequence | (μM) | 100 μM |
| 10 | (Ac)AERFYECCKEP-amide | >10 | 15 |
| 11 | (Ac)SARFYECCKEP-amide | >10 | 13 |
| 1 | (Ac)SEAFYECCKEP-amide | 0.277 | 70 |
| 12 | (Ac)SERAYECCKEP-amide | >10 | 14 |
| 13 | (Ac)SERFAECCKEP-amide | >10 | 18 |
| 14 | (Ac)SERFYACCKEP-amide | >10 | 14 |
| 15 | (Ac)SERFYEACKEP-amide | >10 | 19 |
| 2 | (Ac)SERFYECAKEP-amide | 0.165 | 85 |
| 16 | (Ac)SERFYECCAEP-amide | >10 | 19 |
| 3 | (Ac)SERFYECCKAP-amide | 0.426 | 75 |
| 17 | (Ac)SERFYECCKEA-amide | <10 | 59 |
| 25 | (Ac)NGEWDLVGIPGKRSERFYECCKEPYPD | 0.362 | 90 |
| | VTFTV-amide | | |
| 18 | (Ac)GIPGKRSERFYECCK-amide | ND, NA | |
| 4 | (Ac)GIPGKRSERFYECCK-amide | 1.21 | 73 |
| 5 | (Ac)IPGKRSERFYECCKEPYP-amide | 0.71 | 79 |
| 6 | (Ac)IPGKRSERFYECCKEPYPDV-amide | 0.626 | 73 |
| 19 | SERFYECCKEP | >10 | 24 |
| 7 | (Ac)SERFYECCKEP-amide | 0.125 | 89 |
| 8 | (Ac)YECCKEPYPDVTFTVTMR-amide | 1 | 65 |
| 9 | (Ac)SERFYECCKEP-amide | 0.316 | 81 |
| 20 | (Ac)YECC-amide | <10 | 62 |
| 21 | (Ac)HSVTYSCCPDT-amide | >10 | 10 |
| 22 | (Ac)NTRKYECCAEI-amide | >10 | 16 |
| 23 | (Ac)NTRKYECCAEI-amide | >10 | 16 |
| 24 | (Ac)AVGTYNTRKYECCAEIYPDI-amide | >10 | 15 |
| 26 | (Ac)FYEC | <10 | 54 |
| 28 | (Ac)ECCK-amide | | 87 |

### EXAMPLE 3

### Fluo-β-Amyloid₁₋₄₂ Binding Assay

Biotinylated huma7 peptide was dissolved in 20 mM HEPES, pH 8.0 and 50 μL [1 μg] pensed into each well of a 96-well NeutrAvidin coated black plate [Pierce] and incubated 25° C. for about 2 hr. Excess peptides were removed by washing three times with Hank's solution. Non-specific binding sites on the plate were blocked by 20 mM HEPES, pH 7.5 containing 0.1% ovalbumin for 30 min at 25° C. The contents were discarded the wells were washed three times with Hank's solution. Inhibitor or test compound was added to the appropriate wells followed by 100 nM of Fluo-β-amyloid1-42 [Perkin Elmer] and incubated for 1 hr at 25° C. Unbound peptides were removed by washings 3 times with Hank's solution containing 0.1% ovalbumin. Bound fluorescence was measured in a LJL fluorescence reader.

Following the procedure described above, representative peptides of the present invention were tested for amyloid-β binding, with results as listed in Table 3.

TABLE 3

Fluo-β-amyloid₁₋₄₂ Binding to Biotin-huma7-5

| | | |
|---|---|---|
| | | % |
| | | Inh @ |
| Seq ID | SEQUENCE | 100 μM |

| | | |
|---|---|---|
| 25 | NGEWDLVGIPGKRSERFYECCKEPYPDVTFTV | 100 |
| 27 | SGYIPNGEWDLVGIPGKRSERFYECCKEPYPD | 90 |
| Aβ₄₂ | | 85 |
| α-BTX | | 100 |

### EXAMPLE 4

### Procedure for ¹²⁵I-α-Bungarotoxin Binding Assay

Biotinylated huma7 peptide was dissolved in 20 mM HEPES, pH 8.0 and 50 μL [1 μg] was dispensed into each well of a 96-well NeutrAvidin coated Flash plate [Pierce] and incubated at 25° C. for about 2 hr. Excess peptides were removed by washing three times with Hank's solution. Non-specific binding sites on the plate were blocked by 20 mM HEPES, pH 7.5 containing 10% fetal bovine serum for 30 min at 25° C. The contents were discarded and the wells were washed three times with Hank's solution. Inhibitor or test compound was added to the appropriate wells followed by 100 pM ¹²⁵I-α-bungarotoxin [Amersham, 2000 Ci/mmol] and incubated for 20 hr. Bound radioactivity was measured in a Topcount [Packard].

Following the procedure described above, representative peptides of the present invention were tested for amyloid-β binding, with results as listed in Table 4.

TABLE 4

¹²⁵I-α-BTX Binding to Biotin-huma7-5

| | | |
|---|---|---|
| Seq ID | SEQUENCE | IC₅₀ (μM) |

| | | |
|---|---|---|
| 10 | AERFYECCKEP | 15 |
| 11 | SARFYECCKEP | 8.4 |
| 1 | SEAFYECCKEP | 9.6 |
| 12 | SERAYECCKEP | >100 |
| 13 | SERFAECCKEP | >100 |
| 14 | SERFYACCKEP | 9.5 |
| 15 | SERFYEACKEP | >100 |
| 2 | SERFYECAKEP | >100 |
| 16 | SERFYECCAEP | 1.3 |
| 3 | SERFYECCKAP | >100 |
| 17 | SERFYECCKEA | >100 |
| 25 | NGEWDLVGIPGKRSERFYEC | 0.1 |
| | CKEPYPDVTFTV | |
| 6 | IPGKRSERFYECCKEPYPDV | 0.2 |
| 7 | SERFYECCKEP | 19 |
| 27 | SGYIPNGEWDLVGIPGKRSE | 0.2 |
| | RFYECCKEPYPD | |
| α-BTX | | 0.1 |
| Aβ₁₂₋₂₈ | | 0.4 |

### EXAMPLE 5

### Procedure for Thioflavin-T [TFT] Amyloid Aggregation Assay

β-Amyloid 1-42 [Palomar Inc] is dissolved in 20 mM HEPES pH 8.5 buffer and dispensed into each well [50 μM in 20 mM HEPES, pH 7.5 containing 0.001% sodium azide] of a black plate [Pierce]. Inhibitor or test compound is added to the appropriate wells followed by TFT [50 μM] and the fluorescence is measured at time intervals in a LJL Fluorescence reader. An increase in fluorescence represents amyloid aggregation.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A beta amyloid binding peptide comprising a sequence selected from the group consisting of

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| SERFYECCKEP, | (SEQ.ID.NO.:9 ) |
| AERFYECCKEP, | (SEQ ID No.:10) |
| SARFYECCKEP, | (SEQ ID No.:11) |
| SERAYECCKEP, | (SEQ ID No.:12) |
| SERFAECCKEP, | (SEQ ID No.:13) |
| SERFYACCKEP, | (SEQ ID No.:14) |
| SERFYEACKEP, | (SEQ ID No.:15) |
| SERFYECCAEP, | (SEQ ID No.:16) |
| SERFYECCKEA, | (SEQ ID No.:17) |
| GIPGKRSERFYECCK, | (SEQ ID No.:18) |
| SERFYECCKEP, | (SEQ ID No.:19) |
| YECC, | (SEQ ID No.:20) |
| HSVTYSCCPDT, | (SEQ ID No.:21) |
| NTRKYECCAEI, | (SEQ ID No.:22) |
| NTRKYECCAEI, | (SEQ ID No.:23) |
| AVGTYNTRKYECCAEIYPDI, | (SEQ ID No.:24) |
| NGEWDLVGIPGKRSERFYECCKEPYPDVTFTV, | (SEQ.ID.NO.:25) |
| FYEC | (SEQ.ID.NO.:26) |
| SGYIPNGEWDLVGIPGKRSERFYECCKEPYPD | (SEQ.ID.NO.:27) |
| and | |
| ECCK. | (SEQ.ID.NO.:28) |

2. A beta amyloid binding peptide as in claim 1 comprising a sequence selected from the group consisting of:

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| SERFYECCKEP, | (SEQ.ID.NO.:9) |
| YECC, | (SEQ.ID.NO.:20) |
| NGEWDLVGIPGKRSERFYECCKEPYPDVTFTV, | (SEQ.ID.NO.:25) |
| FYEC | (SEQ.ID.NO.:26) |
| SGYIPNGEWDLVGIPGKRSERFYECCKEPYPD | (SEQ.ID.NO.:27) |
| and | |
| ECCK. | (SEQ.ID.NO.:28) |

3. A beta amyloid binding peptide as in claim 2 comprising a sequence selected from the group consisting of:

| | |
|---|---|
| SEAFYECCKEP, | (SEQ.ID.NO.:1) |
| SERFYECAKEP, | (SEQ.ID.NO.:2) |
| SERFYECCKAP, | (SEQ.ID.NO.:3) |
| GIPGKRSERFYECCK, | (SEQ.ID.NO.:4) |
| IPGKRSERFYECCKEPYP, | (SEQ.ID.NO.:5) |
| IPGKRSERFYECCKEPYPDV, | (SEQ.ID.NO.:6) |
| SERFYECCKEP, | (SEQ.ID.NO.:7) |
| YECCKEPYPDVTFTVTMR, | (SEQ.ID.NO.:8) |
| SERFYECCKEP, | (SEQ.ID.NO.:9) |
| YECC, | (SEQ.ID.NO.:20) |
| NGEWDLVGIPGKRSERFYECCKEPYPDVTFTV- | (SEQ.ID.NO.:25) |
| amide | |
| and | |
| FYEC. | (SEQ.ID.NO.:26) |

4. The peptide of claim 1 wherein the peptide contains at least one D-isomeric amino acid residue.

5. A fusion protein comprising the β amyloid binding peptide of claim 1 operably linked to another sequence.

6. A method comprising the steps:
(a) contacting a β amyloid peptide with a β amyloid binding peptide of claim 1, and a putitive inhibitory compound for sufficient time to provide a β amyloid/binding peptide complex; and
(b) measuring a change in quantity of β amyloid/binding peptide complex.

7. A diagnostic method comprising the steps:
(a) contacting a biological sample with a β amyloid binding peptide of claim 1 thereby allowing interaction of a beta amyloid peptide with the binding peptide to form a beta amyloid peptide/binding peptide complex;
(b) isolating the β amyloid peptide/binding peptide complex; and
(c) detecting the β amyloid peptide.

8. A diagnostic method comprising the steps:
(a) contacting a biological sample with a β amyloid affinity capture reagent thereby allowing interaction of a beta amyloid peptide with the affinity capture reagent to form a beta amyloid peptide/affinity capture complex;
(b) isolating the amyloid peptide/affinity capture complex; and
(c) detecting the β amyloid peptide with a labeled β amyloid binding peptide of claim 1.


SEQUENCE LISTING (28 sequences)

SEQ ID NO: 1 - protein, 11 aa, Artificial Sequence
  Description of Artificial Sequence Synthetic peptide
SEAFYECCKEP

SEQ ID NO: 2 - protein, 11 aa, Artificial Sequence
  Description of Artificial Sequence Synthetic peptide
SERFYECAKEP

SEQ ID NO: 3 - protein, 11 aa, Artificial Sequence
  Description of Artificial Sequence Synthetic peptide
SERFYECCKAP

SEQ ID NO: 4 - protein, 15 aa, Artificial Sequence
  Description of Artificial Sequence Synthetic peptide
GIPGKRSERFYECCK

SEQ ID NO: 5 - protein, 18 aa, Artificial Sequence
  Description of Artificial Sequence Synthetic peptide
IPGKRSERFYECCKEPYP

SEQ ID NO: 6 - protein, 20 aa, Artificial Sequence
  Description of Artificial Sequence Synthetic peptide
IPGKRSERFYECCKEPYPDV

SEQ ID NO: 7 - protein, 11 aa, Artificial Sequence
  Description of Artificial Sequence Synthetic peptide
SERFYECCKEP

SEQ ID NO: 8 - protein, 18 aa, Artificial Sequence
  Description of Artificial Sequence Synthetic peptide
YECCKEPYPDVTFTVTMR

SEQ ID NO: 9 - protein, 11 aa, Artificial Sequence
  Description of Artificial Sequence Synthetic peptide
SERFYECCKEP

SEQ ID NO: 10 - protein, 11 aa, Artificial Sequence
  Description of Artificial Sequence Synthetic peptide
AERFYECCKEP

SEQ ID NO: 11 - protein, 11 aa, Artificial Sequence
  Description of Artificial Sequence Synthetic peptide
SARFYECCKEP

SEQ ID NO: 12 - protein, 11 aa, Artificial Sequence
  Description of Artificial Sequence Synthetic peptide
SERAYECCKEP

SEQ ID NO: 13 - protein, 11 aa, Artificial Sequence
  Description of Artificial Sequence Synthetic peptide
SERFAECCKEP

SEQ ID NO: 14 - protein, 11 aa, Artificial Sequence
  Description of Artificial Sequence Synthetic peptide
SERFYACCKEP

SEQ ID NO: 15 - protein, 11 aa, Artificial Sequence
  Description of Artificial Sequence Synthetic peptide
SERFYEACKEP

SEQ ID NO: 16 - protein, 11 aa, Artificial Sequence
  Description of Artificial Sequence Synthetic peptide
SERFYECCAEP

SEQ ID NO: 17 - protein, 11 aa, Artificial Sequence
  Description of Artificial Sequence Synthetic peptide
SERFYECCKEA

SEQ ID NO: 18 - protein, 15 aa, Artificial Sequence
  Description of Artificial Sequence Synthetic peptide
GIPGKRSERFYECCK

SEQ ID NO: 19 - protein, 11 aa, Artificial Sequence
  Description of Artificial Sequence Synthetic peptide
SERFYECCKEP

SEQ ID NO: 20 - protein, 4 aa, Artificial Sequence
  Description of Artificial Sequence Synthetic peptide
YECC

SEQ ID NO: 21 - protein, 11 aa, Artificial Sequence
  Description of Artificial Sequence Synthetic peptide
HSVTYSCCPDT

SEQ ID NO: 22 - protein, 11 aa, Artificial Sequence
  Description of Artificial Sequence Synthetic peptide
NTRKYECCAEI

SEQ ID NO: 23 - protein, 11 aa, Artificial Sequence
  Description of Artificial Sequence Synthetic peptide
NTRKYECCAEI

SEQ ID NO: 24 - protein, 20 aa, Artificial Sequence
  Description of Artificial Sequence Synthetic peptide
AVGTYNTRKYECCAEIYPDI

SEQ ID NO: 25 - protein, 32 aa, Artificial Sequence
  Description of Artificial Sequence Synthetic peptide
NGEWDLVGIPGKRSERFYECCKEPYPDVTFTV

SEQ ID NO: 26 - protein, 4 aa, Artificial Sequence
  Description of Artificial Sequence Synthetic peptide
FYEC

SEQ ID NO: 27 - protein, 32 aa, Artificial Sequence
  Description of Artificial Sequence Synthetic peptide
SGYIPNGEWDLVGIPGKRSERFYECCKEPYPD

SEQ ID NO: 28 - protein, 4 aa, Artificial Sequence
  Description of Artificial Sequence Synthetic peptide
ECCK